(19) United States
(12) Patent Application Publication
(10) Pub. No.: US 2026/0115267 A1
(43) Pub. Date: Apr. 30, 2026

(54) METHODS OF USING ANTI-EGF ANTIBODIES TO AUGMENT THE ACTIVITY OF BRAF AND KRAS INHIBITORS

(71) Applicant: In3Bio Ltd., Hamilton (BM)

(72) Inventors: Erik D'Hondt, Bazel (BE); Miguel Angel Molina-Vila, Badalona (ES)

(73) Assignee: In3Bio Ltd., Hamilton (BM)

(21) Appl. No.: 18/704,738

(22) PCT Filed: Oct. 28, 2022
(86) PCT No.: PCT/IB2022/000655

§ 371 Date: Apr. 25, 2024

Related U.S. Application Data
(60) Provisional application No. 63/272,822, filed on Oct. 28, 2021.

(51) Int. Cl. A61K 39/00; A61K 31/437; A61K 31/506; A61K 39/395; A61P 35/00; C07K 16/22; C07K 16/28

(52) U.S. Cl. CPC A61K 39/001131; A61K 31/437; A61K 31/506; A61K 39/3955; A61P 35/00; C07K 16/22; C07K 16/2863; A61K 2039/507; A61K 2039/82

(57) ABSTRACT

Embodiments of the present disclosure are directed to methods for treating and preventing disease conditions, such as cancer, particularly in those individuals who have developed a resistance to, or who are not responsive to, cancer therapies involving v-Raf murine sarcoma viral oncogene homolog B1 (BRAF) or Kirsten rat sarcoma oncogene homolog (KRAS) inhibitors.

### CROSS-REFERENCE TO RELATED APPLICATIONS

This application claims priority to and the benefit of U.S. Provisional Patent Application No. 63/272,822, filed on Oct. 28, 2021, the content of which is incorporated herein by reference in its entirety for all purposes.

### FIELD

Embodiments of the present disclosure are directed to methods for treating and preventing disease conditions, such as cancer, particularly in those individuals who have developed a resistance to, or who are not responsive to, cancer therapies involving v-Raf murine sarcoma viral oncogene homolog B1 (BRAF) or Kirsten rat sarcoma oncogene homolog (KRAS) inhibitors.

### BACKGROUND

Colorectal cancer (CRC) is a major public health problem. It is the third most commonly occurring cancer in men and the second most commonly occurring cancer in women and the fourth cause of cancer death worldwide. The most updated statistics show that there were 1.8 million cases worldwide in 2018. If the cancer is diagnosed at a localized stage, the survival rate is 91%. If it has spread to surrounding tissues or organs, the 5-year survival rate drops to 72%. Treatment has improved by means of identifying v-raf murine sarcoma viral oncogene homolog B1 (BRAF) inhibitors or Kirsten rat sarcoma oncogene homolog (KRAS) mutations as prognostic and predictive tools. Patients that have the BRAF mutation are associated with a short-term survival rate of generally less than two years. In one study, it was found that patients with wild-type KRAS tumor status can be treated with cetuximab, and had a significantly longer median overall survival than patients with KRAS mutations. Although significant advances have been made in elucidating the genomic abnormalities that cause malignant cancer cells, currently available chemotherapy protocols remain unsatisfactory, and the prognosis for the majority of patients diagnosed with CRC is poor. Accordingly, there is an urgent need for treating and preventing CRC.

### SUMMARY

The present disclosure provides that monoclonal or polyclonal antibodies generated against the Epidermal Growth Factor (EGF) ligand may be used in combination with BRAF or KRAS inhibitors to treat colorectal cancer (CRC). In particular, the present disclosure is based on the surprising discovery that monoclonal anti-EGF antibodies or polyclonal anti-EGF antibodies generated in response to an immunogenic polypeptide comprising the EGF ligand, or parts thereof, may overcome or prevent resistance to BRAF or KRAS inhibitors in a patient.

In one aspect, the present disclosure provides for use of an active immunization targeting Epidermal Growth Factor (EGF) comprising an immunogenic polypeptide including an EGF, or portions thereof, and a BRAF inhibitor, characterized in that the use is for the manufacture of a medicament or a composition for treating colorectal cancer (CRC) in a patient, wherein the BRAF inhibitor is administered according to a continuous regimen based on an average daily dose in the range of 10 mg to 150 mg and the active immunization is co-administered according to a therapeutically effective amount repeated thrice, twice or once a week, once in two weeks, once in three weeks or at least once monthly, wherein said medicament or composition causes an immune response to EGF for inhibition of the metabolic pathway activated by EGF-EGFR.

In some embodiments, the BRAF inhibitor is selected from the group consisting of vemurafenib, dabrafenib and encorafenib.

In some embodiments, the CRC is a metastatic form of CRC.

In some embodiments, the BRAF inhibitor is selected from the group consisting of vemurafenib, dabrafenib, encorafenib, and pharmaceutically acceptable salts thereof, and wherein the active immunization targeting EGF is co-administered according to a therapeutically effective amount repeated is administered twice or once a week or once in two weeks to the patient, wherein the patient has acquired resistance to treatment with a BRAF inhibitor selected from the group consisting of vemurafenib, dabrafenib and encorafenib.

In some embodiments, the immunogenic polypeptide is in a therapeutic amount to reduce STAT3 activation.

In one aspect, the present disclosure provides for use of a BRAF inhibitor and a monoclonal anti-Epidermal Growth Factor (EGF) antibody, characterized in that the use is for the manufacture of a medicament or a composition for treating colorectal cancer (CRC) in a patient.

In some embodiments, the BRAF inhibitor is selected from the group consisting of vemurafenib, dabrafenib, encorafenib, and pharmaceutically acceptable salts thereof.

In some embodiments, the EGF immunogenic protein is as set forth in SEQ ID NO:1 or SEQ ID NO:2.

In some embodiments, the EGF immunogenic protein is as set forth in SEQ ID NO:1.

In some embodiments, the EGF immunogenic protein is as set forth in SEQ ID NO:2.

In some embodiments, the EGF immunogenic protein as set forth in SEQ ID NO: 1 is administered to a patient in combination with a BRAF inhibitor, optionally selected from the group consisting of vemurafenib, dabrafenib, encorafenib, and pharmaceutically acceptable salts thereof.

In one aspect, the present disclosure provides a therapeutic composition for reducing resistance to BRAF inhibitors characterized by comprising an immunogenic polynucleotide having the sequence selected from the group consisting of SEQ ID NO: 1 and SEQ ID NO: 2, optionally including an adjuvant or pharmaceutical excipients.

In some embodiments, the composition is characterized by further comprising an adjuvant.

In some embodiments, the composition is characterized by further comprising pharmaceutical excipients.

In some embodiments, the composition is characterized in that the immunogenic polynucleotide results in the inhibition of an EGF/EGFR pathway, optionally wherein the immunogenic protein is in a therapeutic amount to reduce STAT3 activation.

In one aspect, the present disclosure provides a pharmaceutical kit, characterized by comprising:
(i) a first compartment which comprises an effective amount of an anti-Epidermal Growth Factor (EGF) target antibodies and a second compartment which comprises an effective amount of a BRAF inhibitor); or
(ii) a first compartment which comprises an effective amount of a vaccine producing an immune response to EGF and a second compartment which comprises an effective amount of a BRAF inhibitor; or
(iii) a first compartment which comprises an effective amount of a vaccine producing an immune response to EGFR and a second compartment which comprises an effective amount of a BRAF inhibitor.

In one aspect, the present disclosure provides for use of a BRAF inhibitor, characterized in that the use is for preparation of a pharmaceutical kit for treatment of colorectal cancers in a patient in need thereof, wherein the kit comprises a first compartment which comprises an effective amount of a vaccine producing an immune response to EGF and a second compartment which comprises an effective amount of a BRAF inhibitor.

### BRIEF DESCRIPTION OF THE DRAWINGS

The present disclosure is further described in the detailed description which follows, in reference to the noted plurality of drawings by way of non-limiting examples of embodiments of the present disclosure, in which like reference numerals represent similar parts throughout the several views of the drawings, and wherein:

The disclosure will be more fully understood by reference to the detailed description, in conjunction with the following figures, wherein:

FIG. 1 shows a graph depicting the titration of Cetuximab and anti-IN01 measured by absorbance at dilutions of 1/10, 1/25, 1/50 and 1/75 to determine the optimal concentration of cetuximab and anti-IN01. In the initial preparation, Cetuximab, the patient solution, was three times more concentrated than the purified stock of anti-IN01. The final concentration of 125 g/mL in the culture media was selected for both antibodies, cetuximab and anti-IN01 in all experiments.

FIG. 2 shows a graph depicting the effect on cell viability of Encorafenib combination therapy in the presence of EGF in the HT29 cell line. Cells were treated with increasing nanomolar concentrations of Encorafenib in combination with anti-IN01, Cetuximab and control antibody.

FIG. 3 shows a Western blot in duplicate using cells in the HT29 cell line, grown in the presence of EGF, that have been treated with anti-IN01, Cetuximab and Encorafenib and combinations thereof, for 2 hours. C in the first lane stands for a control, non-treated cell line sample. The second lane is the same control cell receiving EGF at 0.05 UM and in combination with EGF, cells were separately treated with antibody (anti-IN01 antibodies recognizing EGF), Cetuximab, Encorafenib, the combination antibody (anti-IN01) and Encorafenib, and the combination Cetuximab and Encorafenib. The Western blot shows the presence or absence of pEGFR_{(Tyr1068)}, EGFR, PAKT_{(Ser473)}, AKT, pERK_{(Thr 202/Tyr 204)}, and ERK after each type of treatment.

FIG. 4 shows a Western blot in duplicate using cells in the HT29 cell line, grown in the presence of EGF, that have been treated with anti-IN01, Cetuximab and Encorafenib and combinations thereof, for 2 hours. C in the first lane stands for control, non-treated cell line sample. The second lane is the same control cell receiving EGF. In combination with EGF, cells were treated separately with antibody (anti-IN01 antibodies recognizing EGF) at 1/25, Cetuximab at 1/75, Encorafenib at 0.05 μM, the combination of antibody (anti-IN01) and Encorafenib, and the combination of Cetuximab and Encorafenib. The Western blot shows the presence or absence of pEGFR_{(Tyr1068)}, EGFR, pPRAS40_{(Thr 246)}, and PRAS40 after each treatment.

FIGS. 5A-B are graphs that show the emergence of resistance to Encorafenib in the HT29 cell line across an incubation time of 3 weeks and measured by the percentage of cell viability. Cells were treated with 5 μM Encorafenib (FIG. 5A) or 10 μM Encorafenib (FIG. 5B) and incubated with EGF at 0.0 ng/mL, 0.1 ng/mL, Ing/mL, and 10 ng/mL. The percentage of positive wells having viable cells rose and leveled off during weeks 1-3, with higher percentages of positive wells associated with higher concentrations of EGF (FIG. 5A). At 10 μM treatment with Encorafenib (FIG. 5B), resistance began at an earlier time point and the percentage of viable cells was substantially lower.

FIGS. 6A-B are graphs that show the emergence of resistance to Encorafenib in the HT29 cell line across an incubation time of 15 weeks and measured by the percentage of cell viability. Cells were incubated with 0.1 ng/mL EGF and treated with the following: Encorafenib at 5 μM, AB (anti-IN01) at 1/10 in combination with Encorafenib at 5 μM, Cetuximab at 1/30 in combination with Encorafenib at 5 μM, Encorafenib at 10 μM, AB (anti-IN01) at 1/10 in combination with Encorafenib at 10 μM, Cetuximab at 1/30 in combination with Encorafenib at 10 μM (FIG. 6A). The treatments with higher concentrations were tested again (FIG. 6B): Encorafenib at 10 μM, AB (anti-IN01) at 1/10 in combination with Encorafenib at 10 μM, Cetuximab at 1/30 in combination with Encorafenib at 10 μM.

FIGS. 7A-B are graphs that show the emergence of resistance to Encorafenib in the HT29 cell line across an incubation time of 15 weeks and measured by the percentage of cell viability. Cells were incubated with 1.0 ng/ml EGF and treated with the following: Encorafenib at 5 μM, AB (anti-IN01) at 1/10 in combination with Encorafenib at 5 μM, Cetuximab at 1/30 in combination with Encorafenib at 5 μM, Encorafenib at 10 μM, AB (anti-IN01) at 1/10 in combination with Encorafenib at 10 μM, Cetuximab at 1/30 in combination with Encorafenib at 10 μM (FIG. 7A). The treatments with higher concentrations were tested again (FIG. 7B): Encorafenib at 10 μM, AB (anti-IN01) at 1/10 in combination with Encorafenib at 10 μM, Cetuximab at 1/30 in combination with Encorafenib at 10 μM. All tests were conducted at 1.0 ng/ml EGF

FIGS. 8A-B are graphs that show the emergence of resistance to 10 μM Encorafenib in the HT29 cell line with an incubation time of 90 days and measured by the percentage of tumor cell-free wells. Cells were incubated with 0.1 ng/mL EGF and treated with Encorafenib at 10 μM, AB (anti-IN01) at 1/10 in combination with Encorafenib at 10 μM, and Cetuximab at 1/30 in combination with Encorafenib at 10 μM (FIG. 8A). In FIG. 8B, cells were incubated with 1.0 ng/ml EGF and treated with Encorafenib at 10 μM, AB (anti-IN01) at 1/10 in combination with Encorafenib at 10 μM, and Cetuximab at 1/30 in combination with Encorafenib at 10 μM.

### DETAILED DESCRIPTION

The present disclosure is based, at least in part, on the discovery that monoclonal or polyclonal antibodies generated against the Epidermal Growth Factor (EGF) ligand may be used in combination with BRAF or KRAS inhibitors to treat colorectal cancer (CRC). In particular, the present disclosure is based on the surprising discovery that monoclonal anti-EGF antibodies or polyclonal anti-EGF antibodies generated in response to an immunogenic polypeptide comprising the EGF ligand, or parts thereof, may overcome or prevent resistance to BRAF or KRAS inhibitors in a patient.

### Overview

Colorectal cancer (CRC) is a major public health problem. It is the third most commonly occurring cancer in men and the second most commonly occurring cancer in women and the fourth cause of cancer death worldwide. The most updated statistics show that there were 1.8 million cases worldwide in 2018. If the cancer is diagnosed at a localized stage, the survival rate is 91%. If it has spread to surrounding tissues or organs, the 5-year survival rate drops to 72%. Treatment has improved by means of identifying v-raf murine sarcoma viral oncogene homolog B1 (BRAF) inhibitors or Kirsten rat sarcoma oncogene homolog (KRAS) mutations as prognostic and predictive tools. Patients that have the BRAF mutation are associated with a short-term survival rate of generally less than two years.

Only patients with tumors that express wild-type KRAS respond to cetuximab. In one study an objective response was recorded in 27 of 66 patients with wild-type KRAS tumor status but in none of the 43 patients with KRAS nutation. Patients with wild-type KRAS tumor status had a significantly longer median overall survival than patients with KRAS mutations. Although significant advances have been made in elucidating the genomic abnormalities that cause malignant cancer cells, currently available chemotherapy remains unsatisfactory, and the prognosis for the majority of patients diagnosed with cancer remains poor.

Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Although treatment of colon cancers with standard cytotoxic chemotherapies has been optimized for efficacy, more recent approaches to CRC therapeutics are based on classification of CRC into molecular subsets based on their distinct oncogene driver. These molecular drivers of CRC can be attacked therapeutically with targeted agents directed against the specific oncogenes.

Most previous chemotherapy drugs for cancer were nonselective in their activity. Although their exact mechanisms of action were varied and complex, they generally worked by damaging cells undergoing mitosis, which is usually more common in malignant tumors than in most normal tissues. Targeted agents are designed to be selective in their effects by modulating the activity of proteins necessary and essential for oncogenesis and maintenance of cancer, particularly enzymes driving the uncontrolled growth, angiogenesis, invasiveness, and metastasis characteristic of malignant tumors. The increased differential activity usually results in fewer troubling side effects for cancer patients, particularly less nausea, vomiting, and death of cells in the bone marrow and gastrointestinal tract, and increased effectiveness against tumor cells.

KRAS plays an important regulatory role in the signal transduction pathways, such as P13-Akt and RAS-RAF-MAPK signaling pathways that are involved in cell proliferation. Mutations in the KRAS gene impair the GTPase activity of the protein. The mutated KRAS causes aberrant and uncontrolled cell growth and cell transformation, promotes cancer metastasis, and increases resistance to chemotherapy and EFGR targeted therapy in many cancer types including CRC. In CRC, KRAS mutations lead to abnormal activation of the RAS/RAF/MEK/ERK signaling pathway.

Since KRAS mutations have a significant impact on the CRC signaling pathways, KRAS gene activation is closely related to both the occurrence and recurrence of CRC. Studies have shown that CRC patients with KRAS mutations have a shorter survival rate than patients with wild-type KRAS. KRAS mutations are not the deterministic factor for CRC but act in combination with other genetic and environmental factors such as patient age, sex, consistent molecular subtypes and tumor staging. KRAS mutations have been suggested to be a predictive marker for non-response to chemotherapy.

At present, a gene that encodes a serine-threonine kinase downstream from KRAS, the mutational status of BRAF is an important factor in prognosis. The BRAF^{V600E} point mutation encodes an active BRAF kinase, which triggers downstream signaling and bypasses EGFR regulation. Studies have demonstrated that BRAF mutation is an adverse predictive factor in metastatic CRC, but it cannot be considered a predictive marker of cetuximab benefit, and should not be used to exclude patients from anti-EGFR therapy.

A promising set of targets for therapeutic intervention in the treatment of CSC includes supporting the use of BRAF/MEK inhibitors that target BRAF^{V600F} mutations in the mitogen-activated protein kinase (MAPK) pathway. The BRAF kinase is an essential step in intracellular signaling which facilitates signal transmission from the cell surface to the nucleus after activation of the epidermal growth factor receptor (EGFR). The BRAF inhibitor encorafenib is a targeted therapy that binds to the mutated BRAF protein within the cell. Although BRAF and MEK inhibitors have been a good target for therapeutic intervention, eventually patients develop resistance to these treatments.

(EGFR) is a member of the HER-kinase axis and has been the target of choice for the development of several different cancer therapies. However, it has been found that the EGF/EGFR pathway is involved in the emergence of resistance to BRAF and MEK inhibitors. The combination of encorafenib with the anti-EGFR antibody cetuximab has been recently approved in BRAF-positive, advanced colorectal cancer. Cetuximab is a monoclonal antibody treatment for metastatic colorectal cancer as well as certain types of head and neck cancers. Instead, vaccination against EGF represents a promising alternative to the administration of monoclonal antibodies against EGFR.

While several new approaches aim to overcome the various mechanisms of resistance that develop against KRAF and BRAK/MEK inhibiting therapies, new approaches to addressing resistance are needed. One of these is vaccination against EGF, a way to disrupt the EGF/EGFR pathway that is involved in developed resistance to KRAF and BRAK/MEK inhibitors.

BRAF activating mutations, which are usually mutually exclusive with KRAS mutations, represent 5-15% of metastatic CRC, and are associated with a poor prognosis in stage II, III, and IV. The most common BRAF mutation is V600E, which is a kinase-activating mutation. While the impact of BRAF mutation status in prognosis is clear, the benefit when using EGFR-directed treatments remains uncertain, because patients develop a resistance to anti-EGFR therapy. After many clinical trials, FOLFOXIRI plus bevacizumab is now considered to be the standard of care for the first line treatment of BRAF-mutant CRC.

When first line treatment fails, the use of second generation BRAF inhibitors in combination regimens are likely to work better than monotherapy. One of these regimens involves tyrosine kinase inhibitors. A kinase inhibitor works by temporarily blocking one kinase, so that other kinase cannot receive the first kinase. Thus a certain concentration of kinase inhibitors has to be reached in order to block the signal from being passed sufficiently often (probably over 90%) in order to stop the kinase from regulating the expression of certain genes.

Since BRAF-mutant cancer cells are highly dependent on MEK/ERK signaling, the combination of a BRAF inhibitor and a MEK inhibitor has shown a slight increased activity in comparison with either agent alone. BRAF inhibition downregulates the negative feedback signals from ERK, resulting in the activation of the EGFR pathway. This may explain the limited action of BRAF inhibitor in monotherapy in BRAF-mutant tumors and would suggest that concomitant EGFR inhibition may overcome this resistance. Other combinations researched have shown limited activity, suggesting that BRAF^{V600E} inhibitors reactivate the EFGR signaling pathway.

The knowledge about acquired mechanisms of resistance is not completely understood, and some possible explanations have been suggested. For example, activations of the PI3K/AKT pathway have been described in patients receiving BRAF inhibitors, in order to keep intracellular signaling via ERK. Another possible mechanism of resistance is overexpression of the hepatocyte growth factor (HGF), or its receptor, MET. These may lead resistance to BRAF inhibitors through the P13/AKT pathway. Additionally, from in vitro models, it has been described that BRAF inhibitors resulted in a feedback activation of EGFR signaling, in order to maintain ERK phosphorylation. This may be a possible new resistance pathway.

Mutations in EGFR correlate with impaired response to immune checkpoint inhibitors and the development of novel immunotherapeutic approaches for EGFR mutant CRC is of particular interest. Immunization against epidermal growth factor (EGF) has shown efficacy in a phase III trial including unselected NSCLC patients, but little was known about the mechanisms involved in the effects of the anti-EGF antibodies generated by vaccination (anti-EGF VacAbs) or their activity in tumor cells with EGFR mutations.

Kirsten rat sarcoma (KRAS) is one of the most frequently mutated oncogenes in CRC, with about 40% of CRC patients harboring activating missense mutations in KRAS. Chemotherapy based on 5-fluorouracil, leucovorin, and oxaliplatin (FOLFOX) remains the standard first-line treatment for advanced CRC. KRAS mutations, especially G12D, are predictive of an inferior response to chemotherapy and a high risk of recurrence. A second method of attack is the use of receptor tyrosine kinase inhibitors (RTK). However, resistance to RTK inhibitors, such as monoclonal antibodies against epidermal growth factor receptor (EGFR) (cetuximab and panitumumab), is a problem caused by many factors. One is that in patients with KRAS-mutant CRC, the upstream signal regulation of KRAS is interrupted by aberrant activation of the KRAS pathway. Because of these factors and others, the development of specific competitive drugs to inhibit KRAS-driven oncogenesis has eluded the field. KRAS is still considered “undruggable”, and treatment of KRAS-mutant CRC remains a challenge.

KRAS activation accelerates dimerization and phosphorylation of its downstream RAF proteins, which induces a cascade of events involving the RAF-MEK-ERK pathway. Ultimately, activation of ERK results in activating transcription factors that regulate genes promoting cell growth and preventing apoptosis. It seems that inhibition of this pathway would be a good strategy, but the RAF-MEK-ERK cascade is not a linear pathway. It has multiple inputs and outputs, and feedback loops that dynamically regulate signaling. Therefore, only almost complete inhibition of the pathway can effectively treat KRAS mutant tumors. Still, the use of inhibitors of this pathway in combination with other KRAS inhibitors could be promising.

### BRAF Inhibitors

Encorafenib is a small molecule BRAF inhibitor that targets key enzymes in the MAPK signaling pathway. This pathway occurs in many different cancers including melanoma and colorectal cancers. It is an orally available RAF kinase inhibitor with potential antineoplastic activity. Encorafenib specifically inhibits RAF kinase, a serine/threonine enzyme in the RAF/MEK/ERK signaling pathway.

Vemurafenib is an inhibitor of the B-raf enzyme developed for the treatment of late-stage melanoma. The name vemurafenib comes from the V600E mutated BRAF inhibition. It interrupts the B-Raf/MEK step on the B-Raf/MEK/ERK pathway, if the B-Raf has the common V600E mutation. Vemurafab only works in melanoma patients whose cancer has V600E BRAF mutation. Melanoma cells without these mutations are not inhibited by vemurafenib; the drug paradoxically stimulates normal BRAF and may promote tumor growth in such cases.

Dabrafenib acts as an inhibitor of the associated enzyme B-Raf, which plays a role in the regulation of cell growth. It is a medication for the treatment of cancers associated with a mutated version of the gene BRAF. Initially approved as a single agent treatment for patients with BRAF V600E mutation-positive advanced melanoma, clinical data demonstrated that resistance to dabrafenib and other BRAF inhibitors occurs within six to seven months. To overcome this resistance, the BRAF inhibitor was combined with the MEK inhibitor trametinib and the combination was approved as an adjuvant treatment for BRAF V600E-mutated stage III melanoma.

### KRAS Inhibitors

Sotorasib is an inhibitor of the RAS GTPase family. It is used to treat non-small-cell lung cancer. It targets a specific mutation, G12C, in the protein K-Ras encoded by KRAS. Sotorasib is the first approved targeted therapy for tumors with any KRAS mutation. Because the G12C KRAS mutation is relatively common in some cancer types—14% of NSCLC patients and 5% of colorectal patients, there have been high expectations for the drug.

Vaccination against EGF constitutes a novel strategy that, contrary to programmed death 1 blockade, is not intended at reversing tumor-induced immunosuppression by activating the T cells. Instead, it aims to stimulate B cells to produce neutralizing antibodies that sequester circulating EGF, thus preventing its binding to EGFR. Vaccination against EGF, also referred to EGF-pathway targeted immunization, is well tolerated, generates few cases of severe adverse effects, and has shown promising results in two trials enrolling unselected advanced NSCLC patients. However, little was known about the molecular and cellular mechanisms involved in the effects of anti-EGF antibodies besides their capability to block ligand binding and phosphorylation of EGFR, or about their differential activity in tumors with EGFR mutations or other genetic alterations. The instant disclosure shows that anti-EGF VacAbs raised in rabbits suppressed the effects of EGF on cell proliferation, cell cycle, and signal transduction pathways in EGFR-mut NSCLC cell lines, particularly in those derived from untreated patients. The concentrations of EGF used in the experiments (10 ng/mL) were close to those reported in human studies, which have a median around 1 ng/mL and show a significant inter-individual variability (11,23). Remarkably, the anti-EGF VacAbs were also found to consistently reduce the levels of pErk1/2 in absence of exogenous EGF not only in PC9 cells, but also in PC9-GR4 cells, where the growth factor did not show significant effects. One of the possible explanations for this observation could be the existence of receptor/ligand feedback loops in the cell lines used. Sera from patients immunized with an anti-EGF vaccine were also shown to efficiently block the activation

of pErk 1/2 by EGF. Control sera from non-immunized patients had little effect on Erk 1/2 but strongly activated Akt, indicating that the blood of healthy individuals contains growth factors that specifically trigger pAkt in EGFR-mut cells. In contrast, the sera from the four immunized patients analyzed were less active in inducing Akt phosphorylation. Significant differences were observed in the potency of the sera from vaccinated individuals to block Erk1/2 phosphorylation and, to a lesser extent, to activate Akt. Being a new therapeutic approach with only a phase III trial completed, the availability of samples from patients vaccinated against EGF was limited, and it was not possible to correlate it with clinical outcomes.

Previously it has been found that EGF significantly reduced the antiproliferative effects of TKIs such as gefitinib, erlotinib, afati-nib, and osimertinib in several EGFR-mut NSCLC cells, both sensitive and resistant to EGFR TKIs. This finding correlated with the results of Western blotting experiments where the levels of pErk1/2 in cells treated with EGFR TKIs were significantly higher if EGF was present.

It is likely that EGFR-mut patients with high EGF levels might have worse outcomes to EGFR TKIs. Increased serum levels of two EGFR ligands, transforming growth factor alpha and amphiregulin, were reported to correlate with worse responses to EGFR TKIs in unselected NSCLC patients. Regarding EGF, in the only study published to date, for 11 EGFR-mut and 21 EGFR-wt NSCLC patients treated with erlotinib, EGF in the serum correlated with shorter progression-free survival. It has been found that the combination of gefitinib, erlotinib, afatinib, and osimertinib with anti-EGF VacAbs showed a stronger antiproliferative effect than the EGFR TKIs alone in the EGFR-mut cell lines tested, a finding that correlated with a consistent decrease in pErk 1/2 (FIG. 78, compare lanes TKI+EGF with TKI+Ab+EGF). The combination was also superior in blocking the anti-apoptotic and G2/M stimulating effects of EGF.

The anti-EGFR monoclonal antibody cetuximab has also been tested in EGFR-mut cell line models. Similarly to anti-EGF VacAbs, cetuximab blocks ligand binding in vitro and has been shown to prevent ligand-induced EGFR, Erk1/2, and Akt phosphorylation in PC9 and H1975 cells. However, it showed a relatively little effect on EGFR downstream signaling in other EGFR-mut lines such as H3255 or DFCILU-011.29 Regarding receptor down-regulation, there seemed to be significant differences between the two antibodies. Cetuximab has been shown to markedly decrease the levels of total EGFR after 1 to 2 hours of incubation in EGFR-mut cells such as PC9, H1975 or H3255, whereas anti-EGF VacAbs did not induce any significant down-regulation of the receptor after 24 hours of incubation. Finally, although cetuximab was reported to amplify the induction of apoptosis and tumor regression in EGFR-wt, head and neck cancer cell lines, and subcutaneous tumors, it failed to enhance the effects of gefitinib in PC9 xenografts.

In contrast, it has been previously found that anti-EGF VacAbs potentiated the antiproliferative activity of EGFR TKIs in PC9 and the rest of EGFR-mut cell lines tested. This potentiating effect reached statistical significance in all cases, with the only exception being PC9-GR4 cells. The fact that the anti-EGF VacAbs target the ligand instead of the receptor and do not induce EGFR down-regulation might offer a possible explanation for the differences found between the effects of cetuximab and anti-EGF antibodies.

It has also been previously found that the addition of the anti-EGF VacAbs significantly delayed the appearance of clones resistant to gefitinib and afatinib in the PC9 cell line. The addition of anti-EGF VacAbs consistently suppressed this TKI-induced STAT3 activation. In contrast, pSTAT3 has been shown to be elevated in head and neck human tumors progressing to cetuximab, suggesting that STAT3 activation is involved in resistance to this drug.

In summary, anti-EGF VacAbs suppressed the effects of EGF and significantly enhanced the antitumor activity of EGFR TKIs in EGFR-mutated NSCLC cell lines. They also blocked STAT3 activation, reduced AXL expression, and delayed the acquisition of resistance.

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments and are not intended to limit the claimed disclosure, because the scope of the disclosure is limited only by the claims. Unless otherwise defined, all technical and scientific terms used herein have the same meaning as commonly understood by one of ordinary skill in the art to which this disclosure belongs.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more.

As used herein, the terms “proteins” and “polypeptides” are used interchangeably herein to designate a series of amino acid residues connected to the other by peptide bonds between the alpha-amino and carboxy groups of adjacent residues. The terms “protein”, and “polypeptide”, which are used interchangeably herein, refer to a polymer of protein amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof.

Thus, exemplary polypeptides or proteins include gene products, naturally occurring proteins, homologs, orthologs, paralogs, fragments and other equivalents, variants, fragments, and analogs of the foregoing.

As used herein the term, “Antibody” includes any immunoglobulin molecule that recognizes and specifically binds to a target, such as a protein, polypeptide, peptide, carbohydrate, polynucleotide, lipid, etc., through at least one antigen recognition site within the variable region of the immunoglobulin molecule. As used herein, the term is used in the broadest sense and encompasses intact polyclonal antibodies, intact monoclonal antibodies, antibody fragments (such as Fab, Fab′, F(ab′).sub.2, and Fv fragments), single chain Fv (scFv) mutants, multi-specific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgAQ1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as cytotoxics, toxins, radioisotopes, etc. Antibodies can be administered by actively producing them in vivo or passive administering monoclonal antibodies.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgA-1, IgA-2, and etc. The heavy chain constant domains that correspond to the different classes of immunoglobulins are called α, δ, ε, γ, and μ, and respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The terms “full length antibody,” “intact antibody” and “whole antibody” are used herein interchangeably, to refer to an antibody in its substantially intact form, not antibody fragments as defined below. The terms particularly refer to an antibody with heavy chains that contain the Fc region.

“Antibody fragments” comprise only a portion of an intact antibody, wherein the portion preferably retains at least one, preferably most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise on antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population comprise essentially identical amino acid sequence except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “subject”, as used herein, refers to any organism that is capable of developing a bacterial infection. Such organisms include, but are not limited to, human, dog, cat, horse, cow, sheep, goat, mouse, rat, guinea pig, monkey, avian, reptiles, etc.

“Tumor”, as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

The terms “cancer” and “cancerous” refer to or describe the physiological condition in mammals that is typically characterized by unregulated cell growth/proliferation. Examples of cancer include but are not limited to, carcinoma, lymphoma, blastoma, sarcoma, and leukemia. More particular examples of such cancers include colorectal cancer (CRC), squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, “treatment” refers to clinical intervention in an attempt to alter the natural course of the individual or cell being treated and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the disclosure are used to delay development of a disease or disorder.

By “BVN22E or INO1 nucleic acid molecule” is meant a polynucleotide encoding a BVN22E polypeptide. An exemplary BVN22E nucleic acid molecule is reproduced below (SEQ ID NO: 1):

>BVN22E
AATACCGAAAACGATTGCCCTCTGTCTCATGAAGCGTATTGTCTGCACG
ACGGCGTGTGTATGTACATTGAAGCCCTGGACAAATATGCATGTAACTG
TGTCGTGGGCTACGTGGGGGAGCGATGTCAGTTTCGAGACCTGCGTTGG
TGGGATGCGCGCGGCTCGAGCGGTAATACCGAAAACGATTGCCCTCTGT
CTCATGAAGCGTATTGTCTGCACGACGGCGTGTGTATGTACATTGAAGC
CCTGGACAAATATGCATGTAACTGTGTCGTGGGCTACGTGGGGGAGCGA
TGTCAGTTTCGAGACCTGCGTTGGTGGGATGCGCGCGGGGGGTCTGGAG
GTACTAGTGGCGGCGGTGGAGGGTCGGGTACCCCGCAGAACATCACCGA
CCTGTGCGCCGAGTACCACAACACCCAGATCCACACCCTGAACGACAAG
ATCTTCTCGTACACCGAGAGCCTGGCCGATAAGCGTGAAATGGCCATCA
TCACCTTCAAGAACGGTGCGACCTTCCAGGTGGAGGTCCCGGGTAGCCA
GCACATCGATTCACAGAAGAAGGCCATCGAGCGTATGAAGGACACCCTG
CGTATCGCCTACCTGACCGAAGCCAAGGTGGAAAAGCTGTGCGTCTGGA
ACAACAAGACGCCGCACGCCATCGCCGCCATCAGCATGGCCAAT

By “BVN22E or IN01 polypeptide” is meant a polypeptide or fragment thereof having at least about 85%, 90%, 91%, 92%, 93%, 94%, 95%, 96%, 97%, 98%, or 99% amino acid identity (excluding the following amino acid changes: T2S, E3D, N4S, DSE, E11D, A12G, V38I, F44Y, R48K, D51E, and A52L) to the amino acid sequence below (SEQ ID NO:2):

>BVN22E
NTENDCPLSHEAYCLHDGVCMYIEALDKYACNCVVGYVGERCQFRDLRW
WDARGSSGNTENDCPLSHEAYCLHDGVCMYIEALDKYACNCVVGYVGER
CQFRDLRWWDARGGSGGTSGGGGGSGTPQNITDLCAEYHNTQIHTLNDK
IFSYTESLADKREMAIITFKNGATFQVEVPGSQHIDSQKKAIERMKDTL
RIAYLTEAKVEKLCVWNNKTPHAIAAISMAN

A “pharmaceutical excipient” shall mean those commonly utilized within the pharmaceutical art and in particular those found “Handbook of excipients”, (Raymond C. Rowe, Paul J. Sheskey, Paul J. Weller-4th Edition, 2003), the contents of which are incorporated in their entirety.

A “therapeutically effective amount” of a substance/molecule of the disclosure may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Agnew, Chem Intl. Ed. Engl., 33:183-186 (1994)); dynemicin, including dynemicin A; an espmeramicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromefthylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

“Patient response” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of cell proliferation, invasion or metastasis, which may, but does not have to, result in the regression or ablation of a disease lesion; (7) relief, to some extent, of one or more symptoms associated with the disorder; (8) increase in the length of disease-free presentation following treatment; and/or (9) decreased mortality at a given point of time following treatment.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

Signal transducer and activator of transcription 3 (STAT3) is an oncogenic transcription factor that is active in many human cancers and regulates the transcription of several genes that are involved in cell cycle progression, antiapoptosis, cell survival, and angiogenesis.

STAT3 can be activated by EGFR, JAK2, and other tyrosine kinases whose activation can be mediated by EGF, leukemia inhibitory factor (LIF), and other cytokines. Therefore, STAT3 is a convergent point of many signaling pathways and has a major role in oncogenesis and tumor metastasis. It is thought that STAT3 is activated by various forms of mutant EGFR and may contribute to the oncogenic effects of these mutants in various cancer cells (e.g., CRC cells).

Following activation by either ligand binding or mutation, EGFR initiates a cascade of signal transduction pathways that alter the biology of the cell through transcriptional and post-translational mechanisms. The signaling pathways that mediate these changes include the Ras-Raf-mitogen-activated protein (MAP) kinase (MAPK), phosphoinositide 3-kinase-AKT, and signal transducers and activators of transcription (STAT) 3 and STAT5 signal transduction pathways. The STAT families of transcription factors are activated by phosphorylation on a conserved tyrosine residue, leading to dimerization, nuclear translocation, and DNA binding. STAT1, STAT3, and STAT5 are also phosphorylated on a serine residue in their COOH terminus; this phosphorylation it is thought is dispensable for dimerization, nuclear translocation, and DNA binding, but is required for maximal transcriptional activity of some genes.

It has been recently shown that STAT3 is activated by several of these EGFR mutants in a genetically defined system. It is not known which of the signal transduction pathways downstream of mutant EGFR are required to mediate its oncogenic properties, however, given the role of STAT3 in a wide range of human malignancies, and the fact that it is activated by EGF in various cell types, it is believed that STAT3 is necessary for the oncogenic effects of somatic mutant EGFRs. It has been reported that STAT3 is activated in fibroblasts expressing mutant EGFRs, as well as in two NSCLC lines with naturally occurring EGFR mutations, and that this activation is required for the transformation and survival of these cells.

The activation of STAT3 often involves a ligand-receptor interaction. STAT3 can be activated by many various cytokines, including interferons, EGF, G-CSF, and interleukin (IL-6) family cytokines. Binding of cytokines to their cognate receptors leads to JAKs phosphorylation, STAT3 dimerization, nuclear translocation, DNA binding, and gene activation (12, 13). In addition, STAT3 phosphorylation can also be induced by cytoplasmic tyrosine kinase, such as Src family kinase (14). It has been reported that elevated EGFR activity and STAT3 activation is positive correlated in many primary tumor specimens and tumor-derived cell lines, including NSCLC, breast cancer, and head and neck carcinomas.

Increased STAT3 activity is observed in lung adenocarcinomas and cell lines expressing mutant EGFRs. Without being bound to any particular theory, STAT3, it is believed, is required by mutant EGFRs and is necessary for its downstream phenotypic effects. Inhibiting STAT3 function in fibroblasts abrogates transformation by mutant EGFR.

Previous studies suggest mutant EGFR induces activation of gp130/JAK/STAT3 pathway by means of IL-6 up-regulation. Tumor expression of IL-6 and IL-6 receptor components gp80 and gp130 had been found in NSCLC specimens (20). It has also been observed that increased levels of pro-inflammation cytokines such as IL-6 and IL-8 are also associated with NSCLC tumorigenesis and prognosis. These indicate that IL-6 and its downstream pathway are potential to be the target for patient with NSCLC harboring EGFR mutation. However, the mechanism about IL-6 induction by oncogenic EGFR mutations in NSCLC remains unclear; however, it is thought that NF-kB and STAT3 signaling are responsible for regulating IL-6 autocrine in lung cancer.

According to one aspect of the disclosure anti-EGF antibodies are used for treating patients suffering from cancers that may benefit from treatment with a BRAF and/or KRAS inhibitor by administering to the patient in need of such treatment a flexible and active regimen for combining an BRAF and/or KRAS Inhibitor and anti-EGF antibodies according to the disclosure for inhibition of the pathway activated by EGF-EGFR binding (mAb), wherein the BRAF and/or KRAS inhibitor is administered according to a continuous regimen based on an average daily dose in the range of about 10 to 250 mg and the immunogenic EGF protein according to the disclosure is co-administered according to a dosing regimen achieving a therapeutic effective amount repeated thrice, twice or once a week, once in two weeks, once in three weeks or at least once monthly.

According to one aspect of the disclosure anti-EGF antibodies are used for treating patients suffering from cancers that may benefit from treatment with a BRAF and/or KRAS inhibitor by administering to a patient in need of such treatment a flexible and active regimen for combining BRAF and/or KRAS inhibitor and a vaccine producing an immune response to EGF, wherein the BRAF and/or KRAS inhibitor is administered according to a continuous regimen based on an average daily dose in the range of about 10 to 250 mg and the vaccine according to the disclosure is co-administered according to a dosing regimen achieving a therapeutic effective amount repeated thrice, twice or once a week, once in two weeks, once in three weeks or at least once monthly.

The methods of the present disclosure are not limited to the treatment of NSLC. Instead, it will be readily understood that the bio-molecular pathways addressed and BRAF and/or KRAS inhibitors resistance obviated by the methods of the present disclosure may find application in the treatment of other disease conditions; any disease condition in which treatment with an BRAF and/or KRAS inhibitor would result in a beneficial result for a patient under treatment. “Beneficial results” may include, but are in no way limited to, lessening the severity of the disease condition, preventing the disease condition from worsening, curing the disease condition and prolonging a patient's life or life expectancy. These disease conditions may relate to or be modulated by EGFR or any other kinase that may be clinically affected with the methods of the present disclosure.

More specifically, the experimental studies as set forth in the following examples have demonstrated clinical activity of BRAF and/or KRAS inhibitors at the daily dosing regimens in molecular studies on these tumors demonstrated effective inhibition of the EGFR signaling cascade. The examples confirmed that the molecular studies properly reflected the behavior of these BRAF and/or KRAS inhibitors as observed in other model systems. The disclosure also surprisingly demonstrates that BRAF and/or KRAS inhibitors in combination with anti-EGF antibodies, which are passively administered or actively produced by the administration of a vaccine producing such antibodies, can inhibit tumor growth effectively in molecular models—even in tumors that demonstrated a resistance to conventional BRAF and/or KRAS inhibitor therapy.

In one illustrative embodiment the anti-EGF antibodies used in the pre-clinical studies are actively produced by immunizations with BVN22E vaccine as described in PCT application WO 2019/016597 A2 entitled: Synthetic Proteins and Therapeutic Uses Thereof. It is contemplated within the scope of the disclosure that other vaccine formulations that produce an immune response to EGF or EGFR may be used. It is also within the Scope of the disclosure that vaccines producing an immune response to other growth factors, or their receptors may also be used. In particular, immunogenic protein BVN22E as set forth in WO 2019/016597 A2, the content of which is incorporated in its r entirety by reference, may be used to produce anti-EGF antibodies according to the disclosure.

BVN22E has a molecular weight of about 120 kDt, its EGF domain include the region which presents or constrains the β-loop, e.g., the region defined by about cysteine 6 to about cysteine 42, the region defined by about cysteine 6 to about cysteine 31 or the region defined by about cysteine 22 about cysteine 33 or the region defined by about cysteine 22 about cysteine 31 or the region defined by about cysteine 62 about cysteine 14 of the synthetic protein sequence. Without being bound by any particular theory, it is contemplated that different regions or sub-regions between cysteine 6 and cysteine 42 may have beneficial effects when incorporated into the synthetic proteins/molecules. It is thought that the following regions may have beneficial effects: the region between cysteine 6 and cysteine 14, the region between cysteine 6 and cysteine 20, the region between cysteine 6 and cysteine 31, the region between cysteine 6 and cysteine 33, and the region between cysteine 6 and cysteine 42. It is also contemplated that the reverse progressive sequence may also be beneficial. For example, the following regions may have beneficial effects: the region between cysteine 42 and cysteine 33, the region between cysteine 42 and cysteine 31, the region between cysteine 42 and cysteine 20, the region between cysteine 42 and cysteine 14, and the region between cysteine 42 and cysteine 6. It is further contemplated within the scope of the disclosure that specific intervals within the region between cysteine 6 and cysteine 42 may provide beneficial effects when incorporated into the synthetic proteins/molecules of the disclosure (e.g., the region between cysteine 6 and cysteine 14, the region between cysteine 14 and cysteine 20, the region between cysteine 20 and cysteine 31, and the region between cysteine 33 and cysteine 42).

BVN22E and its expressions of the growth factor epitopes fold in a manner allowing their natural conformation to be substantially retained and presented to components of the host immune system in such a way as to elicit a robust host immune response to said epitopes. Examples of suitable natural protein models to model an epitope supporting domain of a synthetic proteins/molecules include, but are not limited to, cholera toxin B sub-unit, E. coli heat-labile LT and LT-II enterotoxin B subunits, veratoxin, pertussis toxin, C. jejuni enterotoxin, Shiga toxin, listeria toxin, tetanus toxoid, diphtheria toxoid, N. meningitidisl outer membrane protein, bacteriophage coat protein, adenovirus and other viral coat proteins. Alternatively, a non-self component of the protein can be small. At a minimum, the non-self sequence(s) should comprise about 9, 10, 11 or more amino acids in length, and include either entirely or in-part at least one human T-cell epitope. Cholera toxin B sub-unit may be used that fulfill the requirements of conferring immunogenicity to the whole protein and allowing appropriate presentation of growth factors, receptors, tumor antigens or epitopes thereof to the host immune system.

BVN22E may be useful in treating chronic diseases, for example, colorectal cancer (CRC), breast, lung, bladder, ovarian, vulva, colonic, pulmonary, brain, colorectal, intestinal, head and neck, and esophagus cancers. As different tumor antigens can be expressed and multiple cellular receptors and growth factors over expressed in the said diseases, the proteins described hereunder can contain one or more different tumor antigens, one or more different receptors or growth factors of one or multiple cellular pathways associated with the disease. These proteins are called multivalent.

BVN22E is a synthetic protein comprised of a homogeneous synthetic proteins/molecules expressing one or more epidermal growth factor (EGF) neutralizing domains (e.g., TSP domains). The protein may be in the form of a synthetic proteins/molecules and may be useful in treating chronic diseases, for example, colorectal, breast, lung, bladder, ovarian, vulva, colonic, pulmonary, brain, colorectal, head and neck, and esophagus cancers. BVN22E is a synthetic proteins/molecules expressing or including synthetic EGF sequences and CT-B sequences. BVN22E contains, a growth factor component of the synthetic protein sequence that includes a sequence that is less than 80% identical to EGF. For example, a growth factor component may include an EGF sequence with 11 amino acid substitutions that may increase the immunogenicity of the growth factor portion of the synthetic protein sequence. Without being bound by theory, it is believed that the region of EGF that ‘presents’ or constrains the B-loop (e.g., the region defined by Cys6 to Cys31) may be an important to include in the synthetic protein and amenable to target for amino acid modification.

BVN22E includes one or more linkers or spacers. One or more of the embodiments described above include sEGF fused to CT-B such that the sEGF portion of the synthetic molecule is separated from the CT-B portion by a GGSGGTSGGGGGSG (SEQ ID NO: 20) linker. These resulting recombinant or chimeric proteins essentially included sEGF fused directly to CT-B. In other embodiments, the EGF and CT-B components of the chimeric protein are effectively separated by 3 to 14 amino acids, which form a flexible spacer or linker between the two domains. It is contemplated that the linker may be 3, 4, 5, 6, 7, 8, 9, 10, 11, 12, 13, or 14 amino acids in length. In some cases in which a growth factor has a larger size (e.g., human growth factor), it may be useful to use a longer linker sequence. The following exemplary linkers may also be used and include, but are not limited to, the following: SSG (SEQ ID NO: 3), SSGGG (SEQ ID NO: 4), SGG (SEQ ID NO: 5), GSSG (SEQ ID NO: 6), GGSGG (SEQ ID NO: 7), GGGGS (SEQ ID NO: 8), SSGGGSGG (SEQ ID NO: 9), SSGGGGSGGG (SEQ ID NO: 10), TSGGGSG (SEQ ID NO: 11), TSGGGGSGG (SEQ ID NO: 12), SSGGGSGGSSG (SEQ ID NO: 13), GGSGGTSGGGSG (SEQ ID NO: 14), SGGTSGGGGSGG (SEQ ID NO: 15), GGSGGTSGGGGSGG (SEQ ID NO: 16), SSGGGGSGGGSSG (SEQ ID NO: 17), SSGGGSGGSSGGG (SEQ ID NO: 18), and SSGGGGSGGGSSGGG (SEQ ID NO: 19). One of skill in the art will appreciate that there are many other sequences/combinations of primarily ‘G’ and ‘S’ that would also serve as useful linker sequences.

It is thought that BVN22E provides significant clinical benefits. For example, BVN22E may be expressed in bacterial systems at commercial scale and purity, while producing stable polypeptides that fold correctly and are functional. Additionally, BVN22E has the advantageous property of requiring much lower levels of protein for vaccination because the amount of carrier necessary significantly lower than prior art methods (e.g., U.S. Pat. No. 5,984,018 to Davila et al.). In this regard, BVN22E is able to deliver more growth factor to a patient in a significantly lower volume of vaccine.

While not wishing to be bound by any theory, it is believed that these suppression of the STAT3 metabolic pathway, which is required for stimulation of the cell signaling pathways responsible for cell proliferation, is aided by BVN22E. It is also believed that the additional inhibition of the STAT3 by the combination dosing regimen of the present disclosure is effective in inhibiting or down-regulating this cell signaling. Moreover, even those patients who are resistant to conventional BRAF and/or KRAS Inhibitor therapy may obtain a beneficial, anti-tumor effect by the combination dosing regimen of the present disclosure, because STAT3 is inhibited as well. The combination therapy of the present disclosure may be associated with hindrance of the disease condition where conventional BRAF and/or KRAS Inhibitor therapies failed. The methods of the present disclosure, therefore, can overcome resistance or non-responsiveness to BRAF and/or KRAS Inhibitor therapy by operating differently than conventional methods at the cellular and molecular level.

In particular embodiments, combination dosage of a BRAF and/or KRAS Inhibitors with anti-EGF antibodies may be effective in treating cancer, and especially colorectal, lung, breast and prostate cancer, in an individual who is resistant to conventional BRAF and/or KRAS Inhibitor therapy. Other forms of cancer that may be treated with the methods of the present disclosure include, but are in no way limited to gastric, colorectal, and ovarian cancer, as well as glioblastoma tumors. Each of these forms of cancer demonstrates significant EGFR expression, making them suitable targets for treatment in accordance with the methods of the present disclosure.

BRAF and/or KRAS Inhibitors suitable for use in accordance with the methods of the present disclosure may include, but are in no way limited to, vemurafenib, dabrafenib, Encorafenib, and Sotorasib within the term “BRAF Inhibitors” or “KRAS Inhibitors.”

The efficacy of a given treatment for cancer can be determined by the skilled clinician. However, a treatment is considered “effective treatment,” as the term is used herein, if any one or all of the signs or symptoms of e.g., a tumor are altered in a beneficial manner or other clinically accepted symptoms are improved, or even ameliorated, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein.

An effective amount for the treatment of a disease means that amount which, when administered to a mammal in need thereof, is sufficient to result in effective treatment as that term is defined herein, for that disease. Efficacy of an agent can be determined by assessing physical indicators of, for example cancer, e.g., tumor size, tumor mass, tumor density, angiogenesis, tumor growth rate, etc. In addition, efficacy of an agent can be measured by a decrease in circulating MIC peptides or fragments thereof in a subject being treated with an agent comprising an antibody or antigen-binding portion thereof as described herein or a nucleic acid encoding an antibody or antigen-binding portion thereof as described herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

This disclosure is further illustrated by the following examples, which should not be construed as limiting.

### Example 1: Anti-EGF Antibodies Significantly Improve the Activity of BRAF Inhibitors in Preclinical Models

Patients who have either the BRAF or MEK mutation and who have been treated with BRAF and MEK inhibitors have been able to achieve a relatively progression-free survival period. After time, patients become resistant to the treatment and this has led to the discovery that the EGF/EGFR pathway is involved in the emergence of resistance to the inhibitors. To combat this, the combination of encorafineb with the anti-EGFR antibody cetuximab has been recently approved in BRAF-positive, advanced colorectal cancer (CRC). The instant disclosure addresses whether antibodies generated by vaccination with IN01 (e.g., anti-IN01 antibodies that recognize EGF) could improve the antitumor activity of encorafenib in BRAF in a colorectal (CRC) cell line, HT29.

The effects of IN01 antibodies, also labeled as Anti-EGF VacAbs. in combination with the following—the BRAF inhibitor, encorafineb and the EGFR antibody cetuximab, and the combination of encorafineb/cetuximab—were tested on the HT29, colorectal ADC cell line with the BRAF^{V600F} mutation and the PIK3CA mutant (p.P449T). All studies and the resulting examples were conducted with anti-EGF VacAbs obtained by immunizing rabbits with recombinant human EGF and then purified. The two-step purification process led to approximately 10-fold dilution of the initial serum collected from the immunized rabbits. All experiments were conducted with further dilutions wherein a 1 to 10 dilution corresponded to 1 to 100 dilution of the original titer.

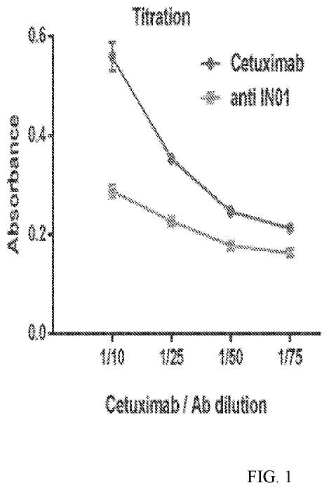

Culture media was prepared with a titration of different concentrations of cetuximab and anti-IN01 to determine the optimal concentration for both antibodies in the media (FIG. 1). In the initial preparation, the patient solution of cetuximab was three times more concentrated the purified stock of anti-IN01. Titration revealed that a concentration of 125 μg/mL was optimal for both cetuximab and anti-IN01.

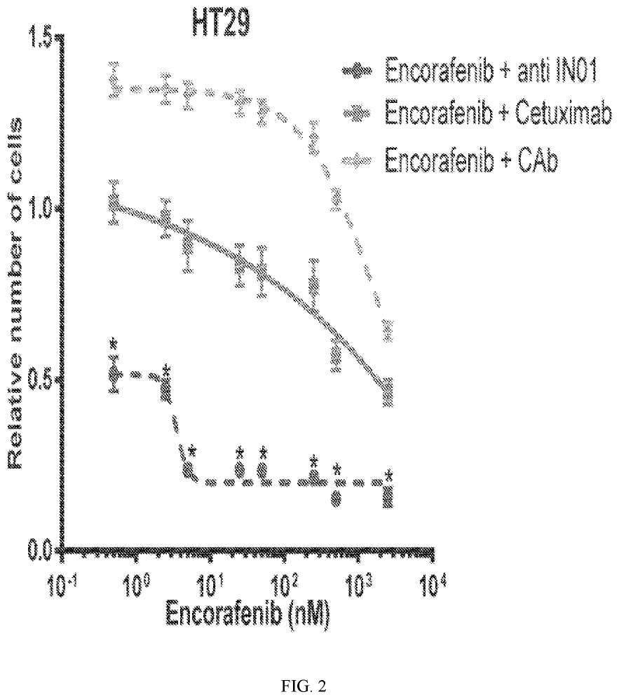

To examine the effects of encorafineb combination therapy on cell viability in the presence of EGF in HT29 cells (FIG. 2), cells were incubated with EGF at a concentration of 10 ng/ML, and treated with increasing nanomolar amounts—10⁻¹, 10⁰, 10¹, 10², 10³, of encorafineb with either anti-IN01, cetuximab, or control antibody (Cab). The data demonstrate that that the combination of encorafineb and anti-IN01 induced a reduction in HT29 cell viability relative to the control antibody combined with encorafineb. Encorafineb and cetuximab induced a reduction in HT29 cell viability, but not as pronounced as encorafineb with anti-IN01.

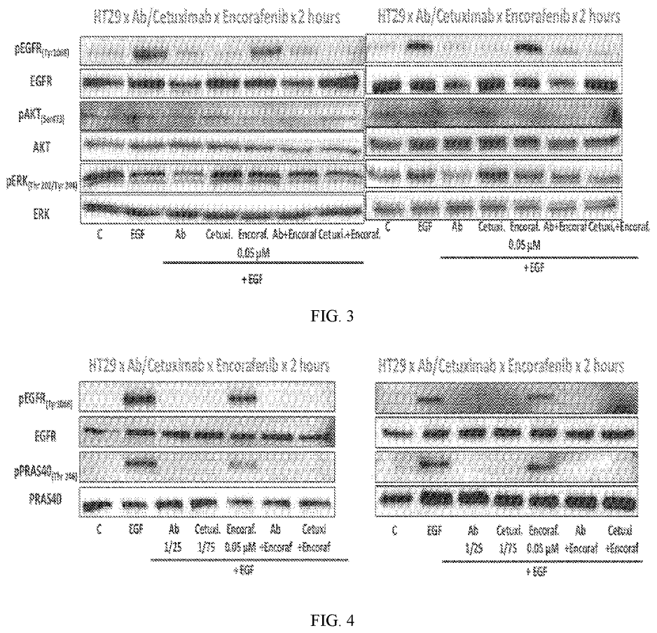

The effects of anti-IN01, alone and in combination with encorafenib was compared to cetuximab alone, encorafenib alone and a combination of cetuximab and encorafenib at a 0.05 μM concentration of each in HT29 cells in the presence of EGF for 2 hours. The changes of total and phosphorylated proteins that indicate cell viability were determined by Western blot in duplicate (FIG. 3) At this concentration, the there was some reduction in signal for pERK, pAKT compared to ERK and AKT for all combinations and inhibitors alone. The most pronounced reduction in signal was between EFGR and pEFGR with the control antibody, anti IN10 alone and in combination with encorafenib, and cetuximab alone and in combination with encorafineb. C in the first lane stands for control, non-treated cell line sample. Second lane is same control cell receiving EGF

The Western blot was repeated with higher concentrations of anti-IN01 and Cetuximab in HT29 cells in the presence of EGF for 2 hours. The changes of total and phosphorylated proteins indicating cell viability were more pronounced with the concentrations of anti-IN01 at 1/25, cetuximab at 1/75, encorafenib at 0.05 μM and combinations of anti-IN01 with encorafenib, and cetuximab and encorafenib (FIG. 4). Total EGFR and PRAS40 were not inhibited, but the phosphorylated form was completely inhibited by anti-IN01, alone and in combination with encorafenib. Encorafenib alone did not inhibit pPRASO, but the combination of cetuximab and encorafenib did. C in the first lane stands for control, non-treated cell line sample. Second lane is same control cell receiving EGF.

### Example 2: Emergence of Resistance to Encorafenib in HT29 Cells is Significantly Reduced by Anti-IN01 in Combination with Encorafenib

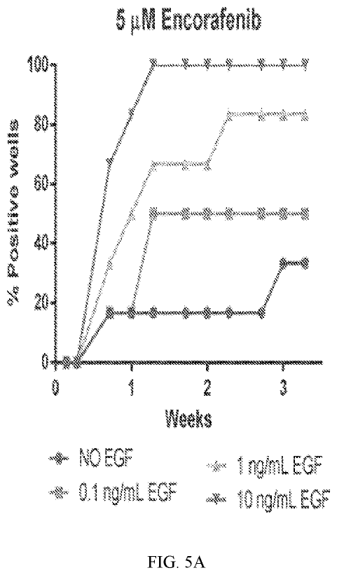

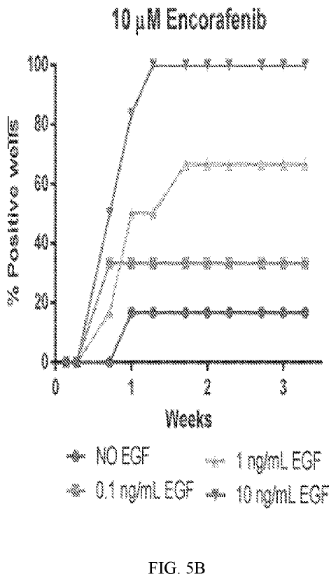

The effect of cells incubated with 5 μM encorafenib and treated with different concentrations of EGF were tested for cell viability during 3 weeks of incubation. In FIG. 5A, cells treated with no EGF showed the lowest proliferation. Cells with 0.1 ng/ml, 1.0 ng/ml and 10 ng/mL EGF showed corresponding higher proliferation as concentrations were increased. FIG. 5B shows the effect of cells incubated with 10 μM and treated with the same concentrations of EGF as in FIG. 5A. The same effects on proliferation were observed, but the doubled concentration of encorafenib corresponded to a lower proliferation of cells than with 5 μM encorafenib, but not by much, suggesting that resistance to encorafenib depends on the concentration. Notably resistance to encorafenib does not reduce by half when the concentration is doubled.

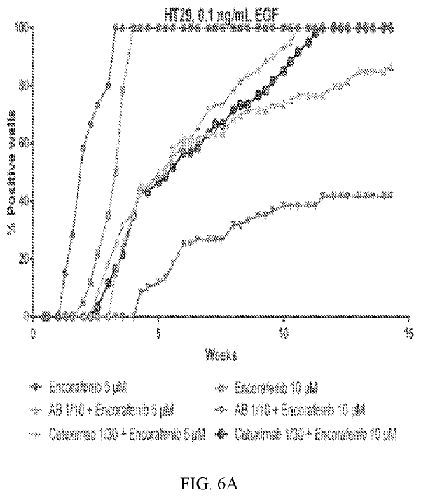

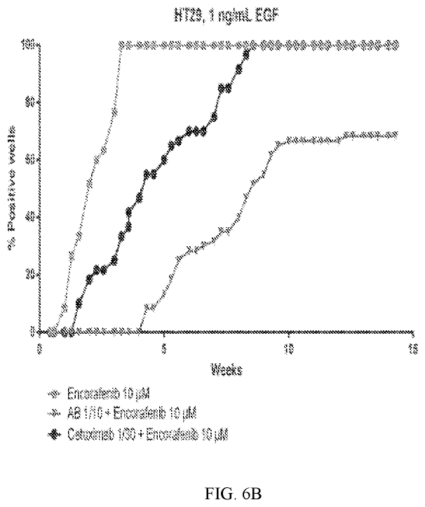

Next, the effects on cells incubated with 0.1 ng/mL EGF were tested. Cells were treated with encorafenib at 5 μM alone and in combination with anti-IN01 at 5 μM and in combination with cetuximab at 5 μM and tested over a period of 15 weeks (FIG. 6A). Cells were also treated with encorafenib at 10 μM alone and in combination with anti-IN01 at 10 μM and in combination with cetuximab at 10 μM (FIG. 6A-B). C The data from the 10 μM concentration were graphed separately (FIG. 6B) because they show that the combination of anti-IN01 and encorafineb in the presence of EGF at 0.1 ng/mL is surprisingly robust against the growth of cancer cells. Encorafenib alone or in combination with cetuximab allows the growth of positive cells to a much greater extent.

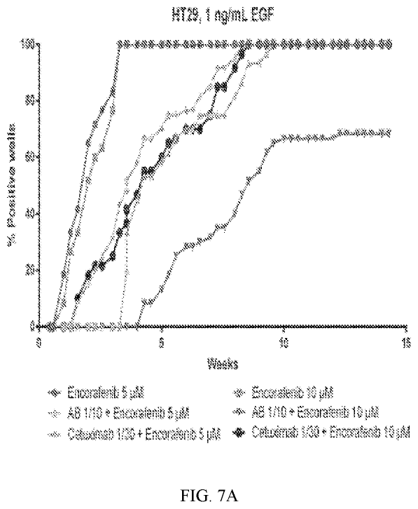

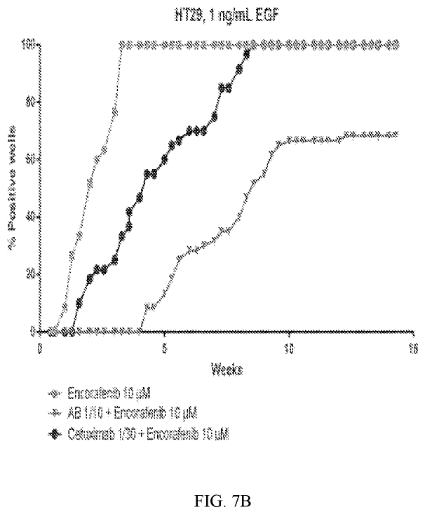

The effects on cells incubated with 1.0 ng/mL EGF were tested over 15 weeks. The same conditions were repeated as in FIGS. 6A-B with the only difference being the 10-fold increase in EGF (FIG. 7A). EGF enhances the growth of cells, so there was a corresponding increase in the number of cells positive for cancer with each set of conditions, indicating resistance to encorafineb alone or in combination with cetuximab. Again, the most robust combination was anti-IN01 with encorafenib, which showed the least amount of positive growth. These data suggest that anti-IN01, or Anti-EGF VacAbs suppressed the EGF proliferation of cancer cells and blocked the EGFR signaling pathways, more efficiently than cetuximab.

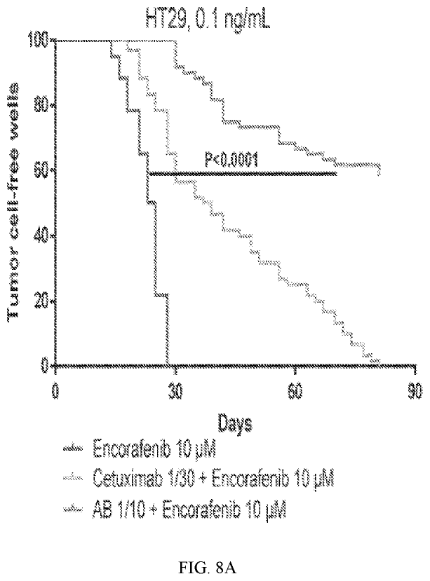

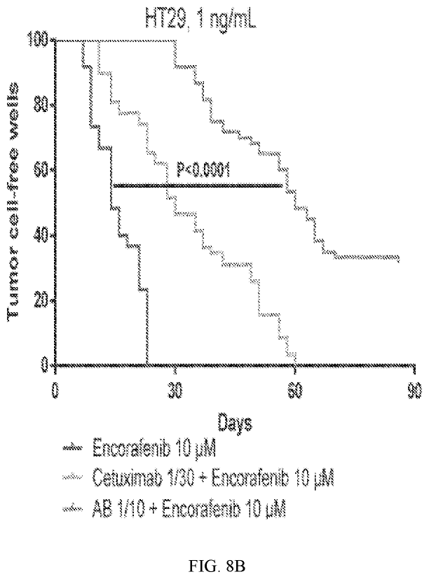

Lastly, the effects of HT29 cells incubated with 0.1 ng/ml and 1.0 ng/mLEGF were tested over a course of 90 days (FIGS. 8A-B). Cells were treated with encorafenib at 10 μM, cetuximab 1/30 with encorafenib at 10 μM, and anti-IN01 1/10 with encorafineb at 10 μM. The antitumor effects of anti-IN01, or anti-EGF VacAbs in combination with encorafineb were superior to those of cetuximab in combination. The addition of anti-IN01, or anti-EGF to the culture medium significantly delayed the emergence of resistant clones to encorafenib in HT29. The effect was more pronounced than the delay observed when adding cetuximab.

### REFERENCES

1. Codony-Servat J., Garcia-Roman S., Molina-Vila M A, et al.
2. Codony-Servat J, Garcia-Roman S, Molina-Vila M A, et al. Anti-Epidermal Growth Factor Vaccine Antibodies Increase the Antitumor Activity of Kinase Inhibitors in ALK and RET Rearranged Lung Cancer Cells. Transl. Oneal. 2020.

What is claimed is:

1. Use of an active immunization targeting Epidermal Growth Factor (EGF) comprising an immunogenic polypeptide including an EGF, or portions thereof, and a BRAF inhibitor or a KRAS inhibitor, characterized in that the use is for the manufacture of a medicament or a composition for treating colorectal cancer (CRC) in a patient,
wherein the BRAF inhibitor is administered according to a continuous regimen based on an average daily dose in the range of 10 mg to 150 mg and the active immunization is co-administered according to a therapeutically effective amount repeated thrice, twice or once a week, once in two weeks, once in three weeks or at least once monthly,
wherein said medicament or composition causes an immune response to EGF for inhibition of the metabolic pathway activated by EGF-EGFR.

2. The use according to claim 1, wherein the BRAF inhibitor is selected from the group consisting of vemurafenib, dabrafenib and encorafenib.

3. The use according to claim 1, characterized in that the CRC is a metastatic form of CRC.

4. The use according to claim 1 or 3, wherein the BRAF inhibitor is selected from the group consisting of vemurafenib, dabrafenib, encorafenib, and pharmaceutically acceptable salts thereof, and wherein the active immunization targeting EGF is co-administered according to a therapeutically effective amount repeated is administered twice or once a week or once in two weeks to the patient, wherein the patient has acquired resistance to treatment with a BRAF inhibitor selected from the group consisting of vemurafenib, dabrafenib and encorafenib.

5. The use according to claim 1, wherein the immunogenic polypeptide is in a therapeutic amount to reduce STAT3 activation.

6. Use of a BRAF inhibitor and a monoclonal anti-Epidermal Growth Factor (EGF) antibody, characterized in that the use is for the manufacture of a medicament or a composition for treating colorectal cancer (CRC) in a patient.

7. The use according to claim 7, wherein the BRAF inhibitor is selected from the group consisting of vemurafenib, dabrafenib, encorafenib, and pharmaceutically acceptable salts thereof.

8. The use according to claim 1, wherein the EGF immunogenic protein is as set forth in SEQ ID NO:1 or SEQ ID NO:2.

9. The use according to claim 8, characterized in that the EGF immunogenic protein is as set forth in SEQ ID NO:1.

10. The use according to claim 8, characterized in that the EGF immunogenic protein is as set forth in SEQ ID NO:2.

11. The use according to claim 9, characterized in that the EGF immunogenic protein as set forth in SEQ ID NO: 1 is administered to a patient in combination with a BRAF inhibitor, optionally selected from the group consisting of vemurafenib, dabrafenib, encorafenib, and pharmaceutically acceptable salts thereof.

12. A therapeutic composition for reducing resistance to BRAF inhibitors characterized by comprising an immunogenic polynucleotide having the sequence selected from the group consisting of SEQ ID NO: 1 and SEQ ID NO: 2, optionally including an adjuvant or pharmaceutical excipients.

13. The therapeutic composition according to claim 12, characterized by further comprising an adjuvant.

14. The therapeutic composition of claim 12, characterized by further comprising pharmaceutical excipients.

15. The therapeutic composition of claim 12, characterized in that the immunogenic polynucleotide results in the inhibition of an EGF/EGFR pathway, optionally wherein the immunogenic protein is in a therapeutic amount to reduce STAT3 activation.

16. A pharmaceutical kit, characterized by comprising:
(i) a first compartment which comprises an effective amount of an anti-Epidermal Growth Factor (EGF) target antibodies and a second compartment which comprises an effective amount of a BRAF inhibitor); or
(ii) a first compartment which comprises an effective amount of a vaccine producing an immune response to EGF and a second compartment which comprises an effective amount of a BRAF inhibitor; or
(iii) a first compartment which comprises an effective amount of a vaccine producing an immune response to EGFR and a second compartment which comprises an effective amount of a BRAF inhibitor.

17. Use of a BRAF inhibitor, characterized in that the use is for preparation of a pharmaceutical kit for treatment of colorectal cancers in a patient in need thereof, wherein the kit comprises a first compartment which comprises an effective amount of a vaccine producing an immune response to EGF and a second compartment which comprises an effective amount of a BRAF inhibitor.


SEQUENCE LISTING (20 sequences)

SEQ ID NO: 1 - DNA, 681 bp, synthetic construct
  misc_feature 1..681: note = artificial
  source 1..681: mol_type = other DNA
aataccgaaaacgattgccctctgtctcatgaagcgtattgtctgcacgacggcgtgtgtatgtacattgaagccctggacaaatatgcatgtaactgtgtcgtgggctacgtgggggagcgatgtcagtttcgagacctgcgttggtgggatgcgcgcggctcgagcggtaataccgaaaacgattgccctctgtctcatgaagcgtattgtctgcacgacggcgtgtgtatgtacattgaagccctggacaaatatgcatgtaactgtgtcgtgggctacgtgggggagcgatgtcagtttcgagacctgcgttggtgggatgcgcgcggcgggtctggaggtactagtggcggcggtggagggtcgggtaccccgcagaacatcaccgacctgtgcgccgagtaccacaacacccagatccacaccctgaacgacaagatcttctcgtacaccgagagcctggccgataagcgtgaaatggccatcatcaccttcaagaacggtgcgaccttccaggtggaggtcccgggtagccagcacatcgattcacagaagaaggccatcgagcgtatgaaggacaccctgcgtatcgcctacctgaccgaagccaaggtggaaaagctgtgcgtctggaacaacaagacgccgcacgccatcgccgccatcagcatggccaat

SEQ ID NO: 2 - protein, 227 aa, synthetic construct
  REGION 1..227: note = artificial
NTENDCPLSHEAYCLHDGVCMYIEALDKYACNCVVGYVGERCQFRDLRWWDARGSSGNTENDCPLSHEAYCLHDGVCMYIEALDKYACNCVVGYVGERCQFRDLRWWDARGGSGGTSGGGGGSGTPQNITDLCAEYHNTQIHTLNDKIFSYTESLADKREMAIITFKNGATFQVEVPGSQHIDSQKKAIERMKDTLRIAYLTEAKVEKLCVWNNKTPHAIAAISMAN

SEQ ID NO: 3 - skipped

SEQ ID NO: 4 - protein, 5 aa, synthetic construct
  REGION 1..5: note = artificial
SSGGG

SEQ ID NO: 5 - skipped

SEQ ID NO: 6 - protein, 4 aa, synthetic construct
  REGION 1..4: note = Artificial Sequence
GSSG

SEQ ID NO: 7 - protein, 5 aa, synthetic construct
  REGION 1..5: note = Artificial Sequence
GGSGG

SEQ ID NO: 8 - protein, 5 aa, synthetic construct
  REGION 1..5: note = Artificial Sequence
GGGGS

SEQ ID NO: 9 - protein, 8 aa, synthetic construct
  REGION 1..8: note = Artificial Sequence
SSGGGSGG

SEQ ID NO: 10 - protein, 10 aa, synthetic construct
  REGION 1..10: note = Artificial Sequence
SSGGGGSGGG

SEQ ID NO: 11 - protein, 7 aa, synthetic construct
  REGION 1..7: note = Artificial Sequence
TSGGGSG

SEQ ID NO: 12 - protein, 9 aa, synthetic construct
  REGION 1..9: note = Artificial Sequence
TSGGGGSGG

SEQ ID NO: 13 - protein, 11 aa, synthetic construct
  REGION 1..11: note = Artificial Sequence
SSGGGSGGSSG

SEQ ID NO: 14 - protein, 12 aa, synthetic construct
  REGION 1..12: note = Artificial Sequence
GGSGGTSGGGSG

SEQ ID NO: 15 - protein, 12 aa, synthetic construct
  REGION 1..12: note = Artificial Sequence
SGGTSGGGGSGG

SEQ ID NO: 16 - protein, 14 aa, synthetic construct
  REGION 1..14: note = Artificial Sequence
GGSGGTSGGGGSGG

SEQ ID NO: 17 - protein, 13 aa, synthetic construct
  REGION 1..13: note = Artificial Sequence
SSGGGGSGGGSSG

SEQ ID NO: 18 - protein, 13 aa, synthetic construct
  REGION 1..13: note = Artificial Sequence
SSGGGSGGSSGGG

SEQ ID NO: 19 - protein, 15 aa, synthetic construct
  REGION 1..15: note = Artificial Sequence
SSGGGGSGGGSSGGG

SEQ ID NO: 20 - protein, 14 aa, synthetic construct
GGSGGTSGGGGGSG